(19) United States
(12) Patent Application Publication
(10) Pub. No.: US 2026/0311335 A1
(43) Pub. Date: Oct. 8, 2026

(54) SYSTEMS AND METHODS FOR GENERATING TISSUE DAMAGE SIGNATURES FROM MEDICAL IMAGES FOR SIMULTANEOUSLY ASSESSING TISSUE DAMAGE EXTENT AND DISTRIBUTION

(71) Applicant: Northwestern University, Evanston, IL (US)

(72) Inventor: Mohammed Elbaz, Chicago, IL (US)

(21) Appl. No.: 19/483,867

(22) PCT Filed: May 13, 2024
(86) PCT No.: PCT/US2024/029118

§ 371 Date: Nov. 12, 2025

Related U.S. Application Data
(60) Provisional application No. 63/501,955, filed on May 12, 2023.

(51) Int. Cl. G06T 7/00; G16H 15/00; G16H 30/40; G16H 50/20; G16H 50/30

(52) U.S. Cl. CPC G06T 7/0014; G16H 15/00; G16H 30/40; G16H 50/20; G16H 50/30; G06T 2207/10081; G06T 2207/10088; G06T 2207/10104; G06T 2207/30048

(57) ABSTRACT

A method for assessing tissue damage, disease, or other medical conditions using medical imaging. Medical image data are accessed with a computer system. The medical image data are processed with the computer system to generate tissue signature data that include one or more probabilistic signature profiles that indicate an extent of tissue damage and distribution patterns associated with the tissue damage. A report that indicates an assessment of tissue damage based on the tissue signature profile is generated with the computer system.

### CROSS-REFERENCE TO RELATED APPLICATIONS

This application claims the benefit of U.S. Provisional Patent Application Ser. No. 63/501,955, filed on May 12, 2023, and entitled “MYOCARDIAL SCAR BURDEN SIGNATURES FOR SIMULTANEOUSLY ASSESSING SCAR EXTENT AND DISTRIBUTION FROM IMAGING DATA,” which is herein incorporated by reference in its entirety.

### BACKGROUND

Myocardial scar is damaged heart tissue that can occur in various heart diseases, causing weakened heart function, irregular heartbeats, heart failure, and even sudden cardiac death. Detecting and measuring the size and distribution of myocardial scar is important for diagnosing and managing heart disease. Medical imaging techniques, such as MRI, CT, PET, or SPECT can be used to show the extent and location of the scar tissue in the heart. Accurate assessment of myocardial scar can help determine the appropriate treatment plan and predict the risk of future heart problems. However, current methods for analyzing myocardial scar from medical image data suffer from limitations that hamper clinical utility, including manual time-consuming scar segmentation hindering reproducibility and non-standardized thresholds leading to unreliable scar extent.

### SUMMARY OF THE DISCLOSURE

It is an aspect of the present disclosure to provide a method assessing tissue damage in a subject using medical imaging. The method includes accessing medical image data with a computer system, where the medical image data include medical images acquired from a subject. The medical images include a calibration region and a target region. The medical image data are processed with the computer system to generate tissue signature data. The tissue signature data include one or more probabilistic signature profiles that indicate an extent of tissue damage, distribution patterns associated with the tissue damage, or both. A report that indicates an assessment of tissue damage based on the tissue signature data is generated with the computer system.

### BRIEF DESCRIPTION OF THE DRAWINGS

FIG. 1 is a flowchart of an example method for generating tissue signature data that includes one or more probabilistic tissue signature values, profiles, and/or indices that quantify tissue damage in a subject based on medical images acquired from the subject.

FIG. 2 is a flowchart of an example method for generating classified feature data using a trained machine learning model that receives tissue signature data as an input to generate the classified feature data as an output that indicates an assessment of a particular disease or medical condition in the subject.

FIG. 3 is a flowchart of an example method for training a machine learning model to receive tissue signature data as input data in order to generate classified feature data as output data, where the classified feature data are indicative of an assessment of a particular disease or medical condition in the subject

FIGS. 4A-4C illustrate different stages of tissue signature profile and index generation for an example of assessing myocardial scar using the methods described in the present disclosure. Stage I (FIG. 4A) computes cross-disparity signature profile between myocardial wall and blood pool by encoding the profile as probability density function from intensity co-disparity associations. Stage II (FIG. 4B) computes an auto co-disparity signature of myocardial wall to encode the unique composition of image intensity distribution patterns in the entire myocardial wall. Stage III (FIG. 4C) computes a threshold-free scar burden signature index (SBSI) that encodes both scar/tissue extent and distribution patterns simultaneously based on probability density distribution differences of scar signature profiles from Stages I and II.

FIGS. 5A-5B show example cases from patients who present with high and low scar burden signature index (SBSI) values. The cases are presented with corresponding magnetic resonance images and their corresponding scar signature profiles.

FIGS. 6A-6D illustrate data plots of example comparisons of SBSI and Troponin in MI patients and control patients.

FIGS. 7A-7D illustrate data plots of example correlations of SBSI with MI % (FIG. 7A), SBSI with Troponin (FIG. 7B), SBSI with left ventricular ejection fraction (LVEF) (FIG. 7C) and MI % with LVEF (FIG. 7D).

FIG. 8 is a block diagram of an example system for assessing myocardial infarction (e.g., scar extent and distribution) using a scar burden signature index generated from medical imaging data.

FIG. 9 is a block diagram of example components that can implement the system of FIG. 8.

FIG. 10 is a block diagram of an example MRI system that can implement some examples described in the present disclosure.

### DETAILED DESCRIPTION

Described here are systems and methods for accurately measuring the severity of tissue damage, or other anomalies or pathological conditions in tissue, using medical imaging techniques such as MRI, CT, PET, SPECT, and/or ultrasound. The systems and methods described in the present disclosure determine and analyze tissue damage signatures (e.g., tissue signature data) to comprehensively measure tissue damage in terms of both extent and distribution patterns, simultaneously. The tissue signature data can include cross-disparity signatures, co-disparity signatures, and/or tissue damage signature indices.

Advantageously, the entire image dimensionality of the medical image data acquired from a subject is employed to probabilistically encode the unique profile of millions of voxel intensity disparities throughout the imaged tissue volume (e.g., heart chamber volume, liver volume, brain volume, etc.). An index called the tissue damage signature index is introduced and used for comprehensive assessment of composite tissue damage. When used in the context of evaluating myocardial scar burden, the signature index may be referred to as a myocardial scar burden signature index (SBSI), which can be used for the comprehensive assessment of scar burden.

As described above, the disclosed systems and methods receive medical images acquired from a subject and process those medical images to generate one or more probabilistic signatures, profiles, or indices, which can be stored as tissue signature data. As a non-limiting example, the tissue signature data can include one or more of cross-disparity signature data, co-disparity signature data, and tissue damage signature indices.

In a first stage of an example workflow, cross-disparity signature data may be generated from medical images of the subject. The cross-disparity signature data can include cross-disparity values, one or more cross-disparity signature profiles, and the like.

To generate the cross-disparity signature profile, voxel index vectors A and B are first defined as two one-dimensional (1D) vectors that carry voxel indices of image data acquired from a reference, or calibration, region (region A) and a region containing target tissues (region B):

$$A = {[ {a}_{1} , {a}_{2} , … , {a}_{m} ]}^{T} ; \qquad ( 1 )$$

$$B = {[ {b}_{1} , {b}_{2} , … , {b}_{k} ]}^{T} ; \qquad ( 2 )$$

where T is the transpose operator, m and k are the number of voxels within the segmented calibration regions and target tissue regions over all image slices, respectively. Although examples are provided with respect to transforming 2D image data into 1D image intensity vectors for processing, it will be appreciated that the methods described herein can be scalable to any data dimension (e.g., 1D, 2D, 3D, . . . , ND).

As a non-limiting example, the calibration region may be a blood pool, such as a left ventricular (LV) blood pool, or a blood pool corresponding to another heart chamber. The target tissue region may be, for example, a region containing myocardial wall tissue, such as LV myocardial wall tissue, or myocardial wall tissue in another region of the heart where tissue damage is being evaluated. In this way, the disclosed systems and methods can be applicable to different chambers of the heart and arteries, thereby providing a comprehensive assessment of the subject's heart health. As a result, the tissue signature data can be used to help assess scar, fibrosis, or other tissue damage of different chambers of the heart and/or arteries (e.g., left ventricle, right ventricle, left atrium, right atrium, aorta, mitral valve, etc.) by appropriate selection of the target region.

As another example, the target region may be a region containing liver tissue under evaluation for fibrosis or other liver damage and the calibration region may be a region containing normal or healthy liver tissue. As another example, the target region may be a region containing brain tissue (e.g., gray matter, white matter, or both) and the calibration may be a region containing normal or healthy brain tissue, cerebrospinal fluid (CSF), or the like. In these instances, the tissue signature data may indicate tissue damage in the brain (e.g., tumor, neurological lesions, amyloid plaque, etc.). As yet another example, the target region may be a region containing tissue under evaluation for a high degree of damage (e.g., scar burden) and the calibration region may be a region of tissue known to have a lower degree of damage (e.g., a lower degree of scar burden).

The calibration region can be any suitable region containing image data that can be used as a calibration reference. In general, the calibration region can be selected as a region containing image data with a noise profile that can be used for calibrating the various indices computed when using the methods described in the present disclosure. The calibration region may contain normal or healthy tissues, control tissues (e.g., a region with little to no contrast uptake when the image data include contrast-enhanced image data), or the like. In the context of nuclear medicine imaging techniques, the calibration region may include a region with little to no radiotracer uptake.

Two vectors, g_{A} and g_{B}, can then be defined with dimensions m×1 and k×1, respectively, that carry the image intensity values corresponding to the voxel indices in the A and B vectors. As one non-limiting example, two repetition matrices, G_{A} and G_{B}, with dimensions m×k and k×m, respectively, can then be defined with respect to the g_{A} and g_{B} image intensity vectors:

$${G}_{A} = {\underbrace{\{ {g}_{A} , {g}_{A} , … , {g}_{A} \}}}_{k} , an m \times k matrix ; \qquad ( 3 )$$

$${G}_{B} = {\underbrace{\{ {g}_{B} , {g}_{B} , … , {g}_{B} \}}}_{m} , a k \times m matrix . \qquad ( 4 )$$

The pairwise image intensity cross-disparity. Ψ_{AB}, between the calibration and target regions can then be computed as follows:

$${ψ}_{AB} = {G}_{A} ⊙ {G}_{B}^{T} ; \qquad ( 5 )$$

where the operator “⊙” indicates the element-wise product (e.g., the Hadamard product) between two matrices. Alternatively, the cross-disparity can be computed using an operator other than the element-wise product. For example, the cross-disparity can be computed using any suitable function that defines disparity or similarity between the two vectors or matrices. As one example, the cross-disparity can be computed as a summation, division, subtraction, or other operator or linear or nonlinear function that defines similarity or disparity between the two vectors or matrices.

Furthermore, the cross-disparity can also be computed without the repetition matrices, G_{A} and G_{B}. The cross-disparity values can be more generally computed as the disparities of each voxel in the target domain to all other voxels in that domain or in the calibration domain. As an alternative example, the cross-disparity values can be computed relative to a subset of the voxels in the calibration and/or target regions instead of all of the available voxels.

In some implementations, the cross-disparity can be vectorized by converting the matrix into a vector by vertically stacking the columns. The result of this step is a cross-disparity vector, Ψ_{AB}, with length m×k.

In some non-limiting examples, to provide for a standardized comparison, and to auto-calibrate for potentially different image intensity ranges between subjects, the cross-disparity values of Ψ_{AB} can be normalized. For example, the cross-disparity values can be normalized to a unified range [0, 1] as follows:

$${ψ}_{AB} = \frac{{ψ}_{AB} - min ( {ψ}_{AB} )}{max ( {ψ}_{AB} ) - min ( {ψ}_{AB} )} . \qquad ( 6 )$$

The cross-disparity signature profile, S_{AB}, can then be computed as a probability density function of the cross-disparity values:

$${S}_{AB} = pdf ( {ψ}_{AB} , β ) ; \qquad ( 7 )$$

where pdf (···) is a probability density function, which in some instances may be estimated as a normalized histogram of Ψ_{AB} values with β bins. As a non-limiting example, β=100 bins may be used.

The cross-disparity signature profile, S_{AB}, can be defined as a discrete distribution as follows:

$${S}_{AB} = \{ {s}_{1} , {s}_{2} , … , {s}_{β} \} such that {s}_{i} is the ith frequency bin of {S}_{A B} . \qquad ( 8 )$$

In some implementations, it can be advantageous to evaluate the peak, or peaks, in the cross-disparity signature profile. In these instances, a peak point index, p, of the cross-disparity signature profile, S_{AB}, can be defined as:

$$p = {arg max}_{i ∈ \{ 1 , 2 , … , β \}} {S}_{AB} . \qquad ( 9 )$$

The cross-disparity signature profile, S_{AB}, can then be split into two parts with respect to the peak point:

$${S}_{pre - peak} = \{ {s}_{p} , {s}_{p - 1} , … , {s}_{2} , {s}_{1} \} ; \qquad ( 10 )$$

$${S}_{post - peak} = \{ {s}_{p} , {s}_{p + 1} , … , {s}_{β - 1} , {s}_{β} \} . \qquad ( 11 )$$

Note that the bins of Sₚᵣₑ₋ₚₑₐₖ are mirrored to match the order of Sₚₒₛₜ₋ₚₑₐₖ bins for later comparison.

In a second stage of an example workflow, co-disparity signature data may be generated from medical images of the subject. The co-disparity signature data can include co-disparity values, one or more co-disparity signature profiles, and the like.

The target tissue region in the medical image can include normal healthy tissue, tissue damage, and other tissue anomalies. As a non-limiting example, when the target tissue is myocardial wall tissue, the target region may contain scar tissue, normal tissue, and/or microvascular obstructions (MVOs). Each of these components may have different image intensity distributions (e.g., different LGE intensity distributions) and may co-exist in the same subject.

This stage of the workflow computes an auto co-disparity signature of the target tissue, which encodes the unique image intensity distribution patterns of the entire target tissue composition into a standardized probability density function.

Voxel index vectors (A and B), image data vectors (g_{A} and g_{B}), and/or repetition matrices (G_{A} and G_{B}) for the calibration and target regions are defined as above (i.e., in Eqns. (1)-(4)). A repetition matrix, G, with dimensions k×k can then be defined with respect to the target tissue image intensity vector, g_{B}, as follows:

$${G}_{C} = {\underbrace{\{ {g}_{B} , {g}_{B} , … , {g}_{B} \}}}_{k} . \qquad ( 12 )$$

The pairwise auto co-disparity of the target region image intensities with itself, Ψ_{BB}, can then be computed as follows:

$${ψ}_{BB} = {G}_{C} ⊙ {G}_{C} . \qquad ( 13 )$$

Here, only the unique pairwise voxel indices are retained, and double computation of the same pair is eliminated. In some implementations, the cross-disparity can be vectorized by converting the matrix into a vector by vertically stacking the columns. The result of this step is a 1D vector Ψ_{BB} with length k×(k−1)/2.

Although Eqn. (13) uses the element-wise product to compute the co-disparity values, in alternative implementations the co-disparity values can be computed using an operator other than the element-wise product. For example, the co-disparity values can be computed using any suitable function that defines disparity or similarity between the two vectors or matrices. As one example, the co-disparity can be computed as a summation, division, subtraction, or other operator or linear or nonlinear function that defines similarity or disparity between the two vectors or matrices. Like the cross-disparity values, the co-disparity values can also more generally be computed as the disparities of each voxel in the target domain to all other voxels in that domain. As an alternative example, the co-disparity values can be computed relative to a subset of the voxels in the target region instead of all of the available voxels.

Alternatively, the co-disparity values can be computed based on the repetition matrix of the target tissue image intensities, G_{B}. In this example, the following upper triangular matrix is first computed:

$${G}_{B}^{U} = {G}_{B} ⊙ [ \begin{matrix}1 & 1 & … & 1 & 1 \\ 0 & 1 & & & 1 \\ ⋮ & & 1 & & ⋮ \\ 0 & & & 1 & 1 \\ 0 & 0 & … & 0 & 1\end{matrix} ] . \qquad ( 14 )$$

The co-disparity values, Ψ_{BB}, can then be computed as:

$${ψ}_{BB} = {G}_{B} ⊙ {( {G}_{B}^{U} )}^{T} . \qquad ( 15 )$$

The co-disparity values computed using Eqn. (15) can alternatively be computed using an operator other than the element-wise product. For example, the co-disparity values can be computed using any suitable function that defines disparity or similarity between the two vectors or matrices. As one example, the co-disparity can be computed as a summation, division, subtraction, or other operator or linear or nonlinear function that defines similarity or disparity between the two vectors or matrices. The co-disparity values can also more generally be computed as the disparities of each voxel in the target domain to all other voxels in that domain. As an alternative example, the co-disparity values can be computed relative to a subset of the voxels in the target region instead of all of the available voxels.

The co-disparity values, Ψ_{BB}, can then optionally be normalized, such as normalized to the unified range of the cross-disparity values, Ψ_{AB}:

$${ψ}_{BB} = \frac{{ψ}_{B B} - min ( {ψ}_{A B} )}{max ( {ψ}_{A B} ) - min ( {ψ}_{A B} )} . \qquad ( 16 )$$

Note that normalization is relative to Ψ_{AB} (Eqn. (5)), not to Ψ_{BB}. In these instances, the co-disparity range between signatures is unified and auto-calibrated for potential image intensity range differences. Using a probability density function inherently normalizes the signature to target tissue volume (area under the curve=1), enabling consistent comparison between subjects.

The target tissue co-disparity signature profile, S_{BB}, can then be computed as a probability density function of the co-disparity values, Ψ_{BB}, as follows:

$${S}_{BB} = pdf ( {ψ}_{B B} , β ) . \qquad ( 17 )$$

As a non-limiting example, the probability density function may be estimated as a normalized histogram of Ψ_{BB} values with β bins. As a non-limiting example, β=100 bins may be used.

In a third stage of an example workflow, one or more tissue damage signature indices may be generated from the cross-disparity signature data and co-disparity signature data. The tissue damages signature indices can include tissue damage signature index values, and the like. This stage introduces a threshold-free signature index that quantitatively encodes composite tissue damage (e.g., myocardial scar burden) of both tissue damage extent (e.g., scar extent) and distribution patterns (e.g., tissue damage distribution patterns, image intensity distribution patterns). In some instances, a higher tissue damage signature index values indicates higher tissue damage, though in other instances, a lower tissue damage signature index value may indicated higher tissue damage. In the example where the tissue damage is myocardial scar burden, the tissue damage signature index may be referred to as a scar burden signature index (SBSI), with higher values of SBSI indicating higher scar burden and worse myocardial infarction severity.

In general, the tissue damage signature index can be generated based on a distance metric computed between the signature profiles described above. First, a distance metric, or measure, between cross-disparity signature data (e.g., Sₚᵣₑ₋ₚₑₐₖ and Sₚₒₛₜ₋ₚₑₐₖ from Stage I (Eqns. (10) and (11))) can be computed as:

$${D}_{AB} = dist ( {S}_{pre - peak} , {S}_{post - peak} ) . \qquad ( 18 )$$

Additionally, a distance metric, or measure, between the cross-disparity signature, S_{AB}, (Eqn. (8)) and the co-disparity signature, S_{BB}, (Eqn. (16)) can be computed as:

$${D}_{BB} = dist ( {S}_{AB} , {S}_{BB} ) . \qquad ( 19 )$$

As one non-limiting example, the distance metric, dist(···), can be a chi-square (χ²) distribution distance. Additionally or alternatively, the distance metric may be any other suitable distance metric, such as a Euclidean distance, a total variation distance, a Chebyshev distance, a Minkowski distance, a Hamming distance, a cosine similarity, a Jaccard similarity, or the like.

The tissue damage signature index (SI) can then be computed as follows:

$$SI = \frac{{D}_{AB}}{{D}_{BB}} . \qquad ( 20 )$$

In some embodiments, when the target tissue is myocardial wall tissue and the calibration region is a blood pool, or other region for calibrating myocardial wall tissue (e.g., a low scar myocardial wall region, etc.) the signature index may be referred to as a myocardial scar burden signature index (SBSI), since it quantitatively encodes composite myocardial scar burden of both scar extent and distribution patterns.

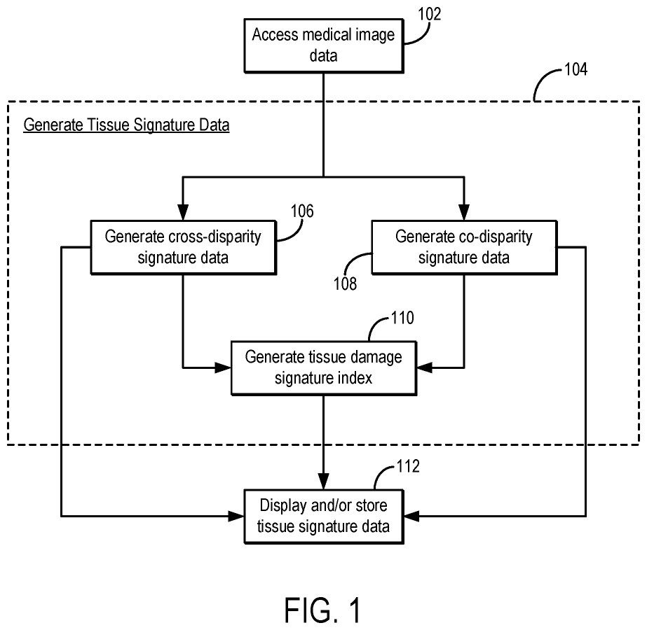

Referring now to FIG. 1, a flowchart is illustrated as setting forth the steps of an example method for generating one or more indices that quantify tissue damage in a subject based on medical images acquired from the subject. Using this method, tissue damage severity is measured using a data-driven, threshold-free probabilistic signature technique that captures both tissue damage extent and unique distribution patterns, simultaneously. Advantageously, the method enables efficient, real-time processing of medical images that encodes unique image intensity profiles (i.e., signatures) by probabilistically encoding a large number of image intensity co-disparities per subject.

The method includes accessing medical image data with a computer system, as indicated at step 102. Accessing the medical image data may include retrieving such data from a memory or other suitable data storage device or medium. Additionally or alternatively, accessing the medical image data may include acquiring such data with a medical imaging system and transferring or otherwise communicating the data to the computer system, which may be a part of the medical imaging system.

In general, the medical image data includes medical images acquired from a subject. The medical images generally include a calibration region containing tissues or other signals that will be used to calibrate signals from damaged tissues when computing the probabilistic signature and indices described above, and a target region containing tissues to be evaluated. The calibration region and the target region can be segmented from the medical images in the medical image data. In some implementations, the segmented calibration and target regions are contained in the medical image data (i.e., the medical images have already been segmented, and accessing the medical image data includes accessing the already segmented calibration and target regions). In other implementations, the medical images can be segmented after accessing the medical image data with the computer system. In these instances, the medical images can be segmented using any suitable segmentation technique, including thresholding based techniques, edge detection based techniques, region-based segmentation techniques (e.g., region growing), techniques based on clustering algorithms (e.g., k-means clustering, Gaussian mixture models (GMMs)), machine learning based techniques (e.g., segmentation using convolutional neural networks), combinations thereof, and so on.

As described above, the calibration region can be a region containing image data (e.g., image intensity values, signals, or other data contained in the medical images) with a noise profile that can be used for calibrating various probabilistic signatures or indices. The calibration region may contain normal or healthy tissues, control tissues (e.g., a region with little to no contrast uptake when the image data include contrast-enhanced image data), or the like. As also described above, the target region may be a region containing tissue under evaluation for tissue damage, and may include heart tissue (e.g., myocardial wall tissue), liver tissue, brain tissue (e.g., gray matter, white matter, or both), or the like. In some instances, the target region may be a region containing tissue under evaluation for a high degree of damage (e.g., scar burden) and the calibration region may be a region of tissue known to have a lower degree of damage (e.g., a lower degree of scar burden).

As one example, the medical image data may include magnetic resonance images acquired with an MRI system. The magnetic resonance images may include images acquired using one or more different pulse sequences to generate magnetic resonance images with different tissue contrast characteristics. In some implementations, the magnetic resonance images can include images acquired using one or more pulse sequences that highlight hyperintensity, hypointensity, or both, in the target tissue regions.

As one example, the magnetic resonance images may include T1-weighted images. As another example, the magnetic resonance images may include T2-weighted images, diffusion-weighted images, perfusion-weighted images, susceptibility-weighted images, proton density-weighted images, or the like. In still other examples, the magnetic resonance images may include images or parameter maps generates from magnetic resonance images, including diffusion parameter maps (e.g., mean diffusivity, fractional anisotropy, etc.), perfusion parameter maps (e.g., cerebral blood volume, cerebral blood flow, mean transit time, etc.), or the like.

The magnetic resonance images may include contrast-enhanced magnetic resonance images, such as those acquired after administering a contrast agent to the subject. The contrast agent may be a gadolinium-based contrast agent, a superparamagnetic iron oxide (SPIO)-based contrast agent, or the like. In a non-limiting example, the magnetic resonance images may include pre-contrast images and post-contrast images acquired after administering a gadolinium-based contrast agent to the subject. The post-contrast images may include images acquired using a late gadolinium enhancement (LGE) MRI acquisition.

Additionally or alternatively, the medical image data may include images acquired with another medical imaging modality. For instance, the medical image data may include images acquired with a computed tomography (CT) system, a positron emission tomography (PET) system, a single photon emission computed tomography (SPECT) system, an ultrasound system, or the like.

Tissue signature data are then generated from the medical image data, as generally indicated at process block 104. As described above, the tissue signature data can include one or more of cross-disparity signature data, co-disparity signature data, or tissue damage signature indices.

Cross-disparity signature data are calculated from the medical image data, as indicated at step 106. The cross-disparity signature data may include one or more cross-disparity values, signatures, profiles, indices or the like. For example, a cross-disparity signature can be computed and encoded as a probability density function by analyzing intensity co-disparities between the calibration and target regions in the medical image data. As a non-limiting example, the cross-disparity signature data can be generated using the methods in Stage I of the example workflow described above.

Co-disparity signature data may also be calculated from the medical image data, as indicated at step 108. The co-disparity signature data may include one or more co-disparity values, signatures, profiles, indices, or the like. For example, a co-disparity signature can be computed and encoded from the medical image data. The co-disparity signature can be an auto co-disparity signature of target region image intensities that captures the unique composition of image intensity distribution patterns in the entire target region, including tissue and tissue damage heterogeneity. As a non-limiting example, the co-disparity signature data can be generated using the methods in Stage II of the example workflow described above.

A tissue damage signature index may in some instances then be computed using the cross-disparity signature data and the co-disparity signature data, as indicated at step 110. For example, the tissue damage signature index can be computed as a threshold-free index that captures both tissue damage extent and distribution patterns, simultaneously. As a non-limiting example, the tissue damage signature index can be generated using the methods in Stage III of the example workflow described above.

The tissue signature data can then be displayed to a user, stored for later use or further processing, or both, as indicated at step 112. Advantageously, the tissue signature data can be used to evaluate the severity of underlying disease or other medical conditions in the subject. The tissue damage signature index described herein, for example, provides a standardized quantitative index of the degree of composite tissue damage severity (e.g., scar severity, fibrosis severity, etc.). Additionally, the tissue damage signature index provides a threshold-free metric, enabling fast and reproducible analysis of large subject cohorts. Advantageously, the tissue signature data also allows for standardized quantitative comparison among different subjects.

In some examples, a report can be generated based on the tissue signature data. The report may include textual information, quantitative or other numerical information (e.g., values, indices, etc.), data plots (e.g., probability density functions), images (e.g., medical images, parameter maps generated from medical images, etc.), and so on. Additionally or alternatively, a report indicating the tissue damage or other health status of the subject can be generated using the tissue signature data.

The disclosed systems and methods have significant clinical implications for various diseases and other pathological conditions. As one non-limiting example, the disclosed system and methods can be used to assess heart diseases to provide a better evaluation of scar severity in different heart chambers. The tissue signature data generated using the disclosed methods can be used to aid in the accurate diagnosis of heart attack (e.g., myocardial infarction) by detecting and assessing the extent of myocardial scar. Additionally or alternatively, the tissue signature data can be used to monitor or assess other heart diseases that involve scar or tissue damage, such as myocarditis, arrhythmia, ventricular tachycardia, atrial fibrillation, cardiac amyloidosis, and cardiomyopathy.

Additionally or alternatively, the tissue signature data can be used to generate a report that indicates potential treatment options for the subject. For example, by quantifying the extent of myocardial scar burden, or other tissue damage, the tissue signature data can be used to help guide the selection of appropriate treatments and interventions. In some instances, the tissue signature data may be used to guide interventions, such as catheter ablation and revascularization procedures. As another example, the tissue signature data can be used for preoperative planning. For instance, the tissue signature data can be used to guide the selection of an optimal surgical approach in complex cardiac conditions by identifying the location and extent of myocardial scar.

As another advantage, the tissue signature data generated using the disclosed methods can be used to identify subjects who are at high risk for adverse health events, such as adverse cardiac events. The tissue signature data can be used to quantify myocardial scar, or other tissue damage, signatures that can help identify patients at higher risk for adverse events (e.g., adverse cardiac events), thereby guiding the selection of appropriate management strategies.

The disclosed systems and methods can also be used to monitoring disease progression and treatment response. For example, the tissue signature data can be generated at multiple time points (e.g., before, during, and after treatment; over the span of days, weeks, months, or years; etc.) to monitor changes in the extent of tissue damage (e.g., myocardial scar) over time. As a result, changes in the tissue signature data over time can be used to assess the effectiveness of interventions, or otherwise monitor the progression of disease. Additionally or alternatively, monitoring the tissue signature data over time can track quality improvement in the subject. By monitoring accurate and reliable measurements of myocardial scar, or other tissue damage, over time, the quality and consistency of care can be monitored and improved as needed.

As yet another advantage, the tissue signature data generated using the disclosed systems and methods can be used to provide personalized medicine or healthcare for the subject. By identifying the location and extent of myocardial scar, or other tissue damage, treatment strategies can be tailored to individual subjects based on their specific disease burden.

In some implementations, the tissue signature data can be used as an input for a machine learning model that has been trained to predict or otherwise identify a disease condition based on one or more of the values, signatures, profiles, or indices stored in the tissue signature data. For example, the tissue signature data provide blue prints for myocardial scar disease state, or other disease states or medical conditions. By training a machine learning model (e.g., a neural network or other suitable machine learning model) using example tissue signatures with appropriate labels (e.g., degree of disease versus associated tissue signature), the disease corresponding to each tissue signature can be predicted or otherwise identified from new incoming patient data.

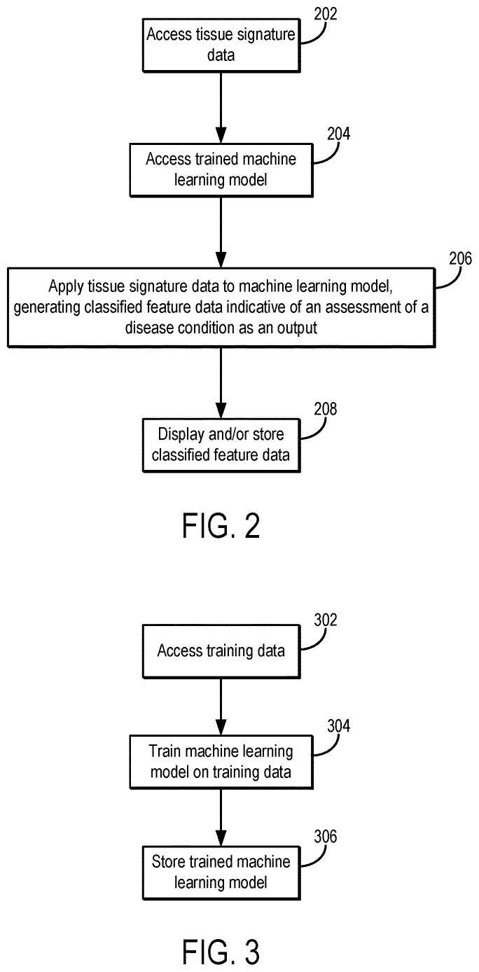

Referring now to FIG. 2, a flowchart is illustrated as setting forth the steps of an example method for generating classified feature data using a suitably trained neural network or other machine learning algorithm. As will be described, the neural network or other machine learning algorithm takes tissue signature data as input data and generates classified feature data as output data. As an example, the classified feature data can be indicative of predicting a particular disease or medical condition in the subject.

The method includes accessing tissue signature data with a computer system, as indicated at step 202. Accessing the tissue signature data may include retrieving such data from a memory or other suitable data storage device or medium. Additionally or alternatively, accessing the tissue signature data may include generating such data using the methods described herein and transferring or otherwise communicating the data to the computer system.

A trained neural network (or other suitable machine learning algorithm) is then accessed with the computer system, as indicated at step 204. In general, the neural network is trained, or has been trained, on training data in order to predict a particular disease or medical condition in the subject based on tissue signature data generated from medical images of the subject.

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network is trained for a classification task, the output layer may include, for example, a number of different nodes, where each different node corresponds to a different classification. For example, a first node may indicate a classification o the subject having a first disease condition, and a second node may indicate a classification of the subject having a second disease condition.

The tissue signature data are then input to the one or more trained neural networks, generating classified feature data as an output, as indicated at step 206. For example, the classified feature data may include a risk score. The risk score can provide physicians or other clinicians with a recommendation to consider additional monitoring for subjects whose tissue signature data indicate the likelihood of the subject suffering from a particular disease or medical condition.

As another example, the classified feature data may indicate the probability for a particular classification (i.e., the probability that the tissue signature data include patterns, features, or characteristics indicative of detecting, differentiating, and/or determining the severity of one or more diseases or medical conditions).

Additionally or alternatively, the classified feature data may classify the tissue signature data as indicating a particular disease or medical condition. In these instances, the classified feature data can differentiate between different diseases or medical conditions. In still other embodiments, the classified feature data may indicate a severity of a disease or medical condition. For example, the classified feature data may include a severity score that quantifies a severity of a disease or medical condition (e.g., a severity score that quantifies the severity of myocardial scar burden).

The classified feature data generated by inputting the tissue signature data to the trained neural network(s) can then be displayed to a user, stored for later use or further processing, or both, as indicated at step 208.

Referring now to FIG. 3, a flowchart is illustrated as setting forth the steps of an example method for training one or more neural networks (or other suitable machine learning algorithms) on training data, such that the one or more neural networks are trained to receive tissue signature data as input data in order to generate classified feature data as output data, where the classified feature data are indicative of predicting a particular disease or medical condition in the subject.

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, and so on.

The method includes accessing training data with a computer system, as indicated at step 302. Accessing the training data may include retrieving such data from a memory or other suitable data storage device or medium. In general, the training data can include tissue signature data generated from one or more populations of subjects. Additionally, the training data may include other data, such as medical image data, electronic health record (EHR) data, or other patient health data. In some embodiments, the training data may include tissue signature data that have been labeled (e.g., labeled as containing patterns, features, or characteristics indicative of one or more medical conditions; and the like).

The method can include assembling training data from the tissue signature data or other relevant data using a computer system. This step may include assembling the tissue signature data into an appropriate data structure on which the neural network or other machine learning algorithm can be trained. Assembling the training data may include assembling tissue signature data and other relevant data. For instance, assembling the training data may include generating labeled data and including the labeled data in the training data. Labeled data may include tissue signature data or other relevant data that have been labeled as belonging to, or otherwise being associated with, one or more different classifications or categories. For instance, labeled data may include tissue signature data that have been labeled as being associated with particular medical conditions.

One or more neural networks (or other suitable machine learning algorithms) are trained on the training data, as indicated at step 304. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as classified feature data. The artificial neural network then compares the generated output with the actual output of the training example in order to evaluate the quality of the classified feature data. For instance, the classified feature data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

The one or more trained neural networks are then stored for later use, as indicated at step 306. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

In an example study, the disclosed systems and methods were used to evaluate myocardial scar burden from LGE MRI data. A database of 100 patients (67 with myocardial infarction and 33 without myocardial infarction) was used in the study. The experiments showed that the tissue signature data (e.g., an SBSI index) is highly correlated with gold-standard scar percent (i.e., scar extent) and Troponin (a blood marker of myocardial injury). Compared to scar percent, the comprehensive SBSI index described in the present disclosure showed a higher correlation with worse ejection fraction (i.e., worse cardiac function), and independent association with markers of cardiac dysfunction and myocardial injury independent of scar percent, suggesting its potential for clinical translation.

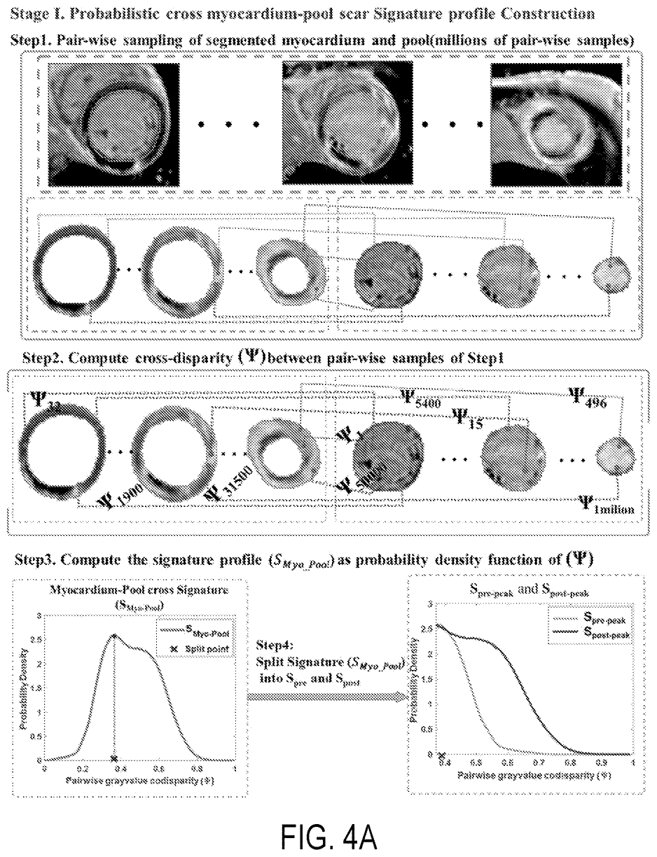

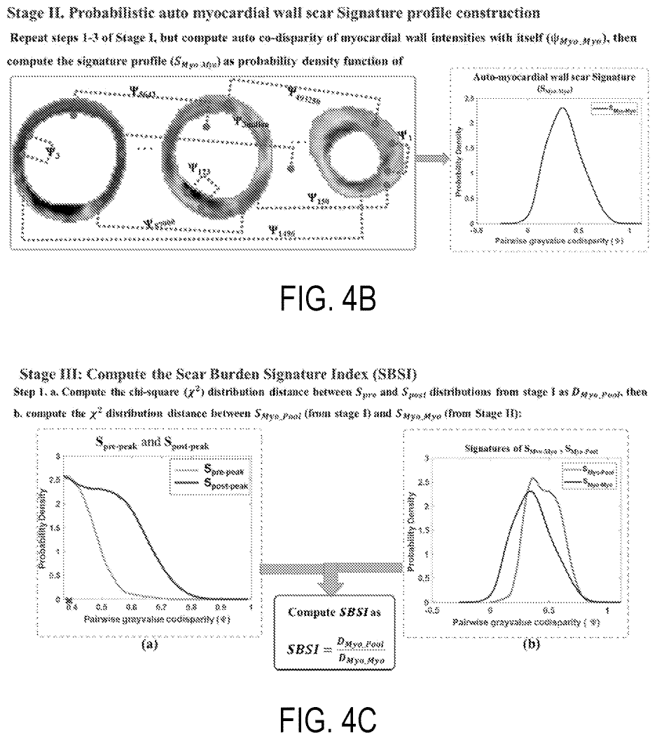

The stages of one example of the methods described in the present disclosure are presented in FIGS. 4A-4C. Stage I (FIG. 4A) computes the unique cross-disparity signature profile between the LGE myocardial wall and blood pool by encoding the profile as probability density function (pdf) from ~10 million intensity co-disparity associations. Given the blood pool is bright, the method is designed mathematically to force higher disparities on the right side of the pdf i.e., post-peak, and lower disparity (normal tissue) on the left side of the pdf peak. Stage II (FIG. 4B) computes an auto co-disparity signature of myocardial wall LGE to encode the unique composition of LGE distribution pattern in the entire myocardial wall, including encoding of scar/tissue heterogeneity. Stage III (FIG. 4C) computes the novel threshold-free scar burden signature index (SBSI) that encodes both scar/tissue extent and LGE distribution patterns simultaneously based on probability density distribution differences of LGE scar signature profiles from Stages I and II.

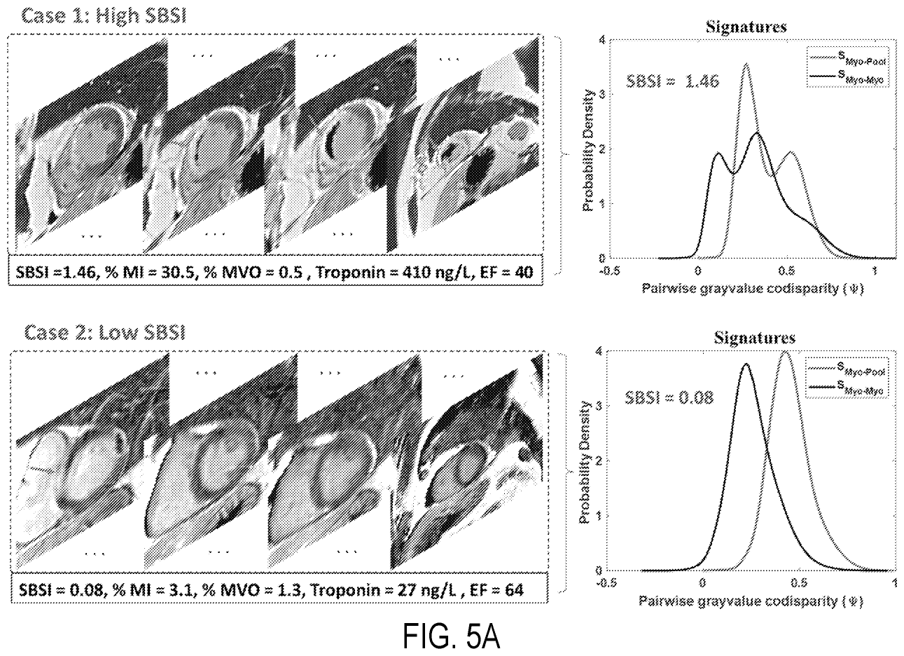

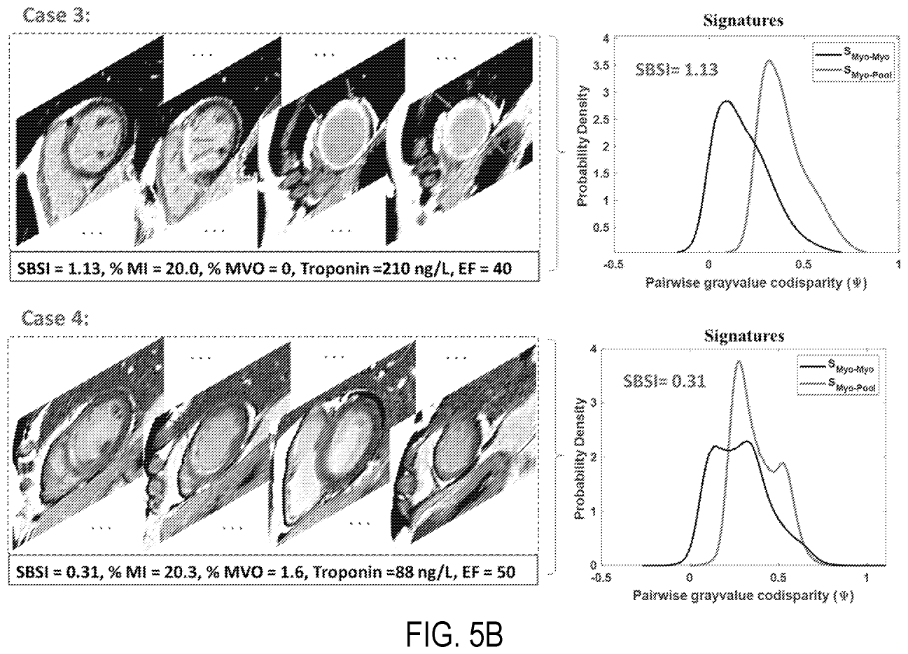

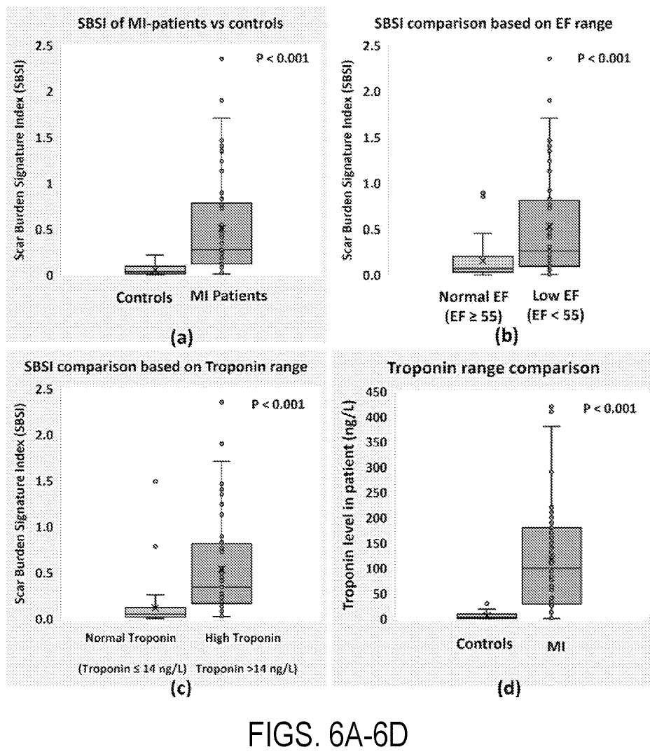

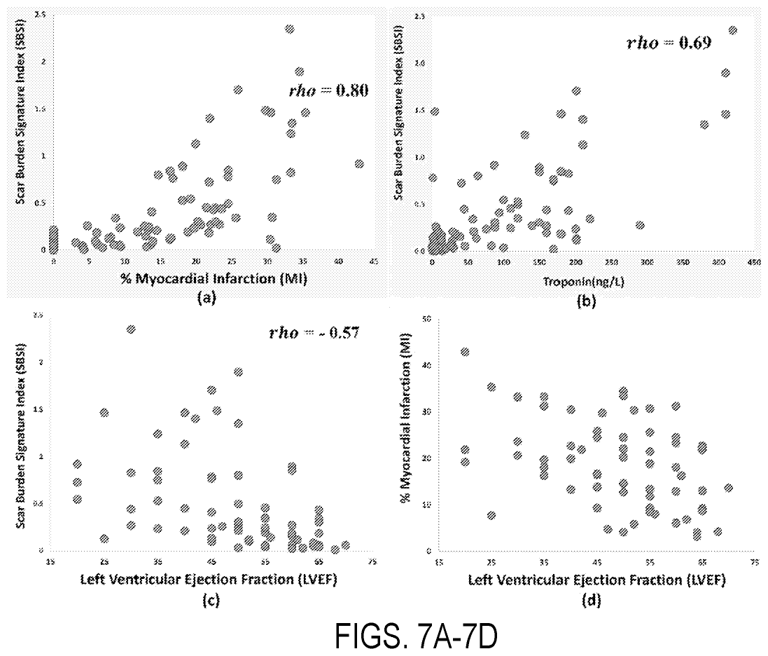

In all 100 analyzed patients, the proposed probabilistic SBSI was successfully computed by encoding 17±11 million LGE pairwise co-disparities per patient. FIG. 5A demonstrates examples of high and low SBSI burden scans. FIG. 5B shows two MI patients with the same scar percentage but ~3.5 times difference in SBSI, which agrees with Troponin levels of ~2.5-fold difference. FIG. 6A shows significantly higher SBSI in MI patients versus controls (p<0.001). SBSI index of scar burden was significantly higher (p<0.001) in patients with elevated Troponin than patients with normal Troponin (FIG. 6C) and in patients with reduced EF (p<0.001) than patients with normal EF (FIG. 6B). A comparison of Troponin range in controls versus MI patients is shown in FIG. 6D. SBSI showed a high correlation with scar extent as measured by scar percentage (Spearman rho=0.80) and moderate correlation to MVO (rho=0.46). SBSI was highly correlated with Troponin biomarker (rho=0.69) (FIG. 7A-7D). In MI patients, SBSI showed higher inverse correlation vs. EF (rho=−0.57) than scar percentage vs. EF (rho=−0.40) and MVO percentage vs. EF (rho=0.02). In multivariate regression, SBSI was associated with Troponin (β=71.84 ng/L, R-squared=0.62, p<0.001) and with EF (β=−9.3%, R-squared=0.14, p=0.01) independent of scar percentage, MVO percentage, and age.

This example study demonstrated the utility of the disclosed systems and methods for quantifying tissue damage (e.g., scar burden) from medical image data (e.g., LGE MRI data). The disclosed tissue signature data combine quantification of tissue damage and distribution patterns in a target tissue (e.g., myocardial wall) through probabilistic analysis of millions of image intensity co-disparities per patient and with real-time processing. Current LGE scar quantification methods rely on tedious manual segmentation or unreliable threshold-based identification of scarred regions, which all lack standardization and reproducibility and ignore LGE distribution patterns and its association to disease severity. Conversely, the disclosed systems and methods are threshold-free, do not require segmentation of scar regions (or other tissue damage regions) and compositely encode both tissue damage extent and distribution patterns.

In some embodiments, the disclosed tissue signature generation techniques can be vectorized with linear complexity allowing real-time processing (e.g., half a second). Advantageously, the tissue damage index probabilistic definition based on encoding signature profiles as probability density functions makes it standardized and inherently normalized to target tissue volume, and a continuous index for standardized comparison between patients with higher index indicating higher tissue damage.

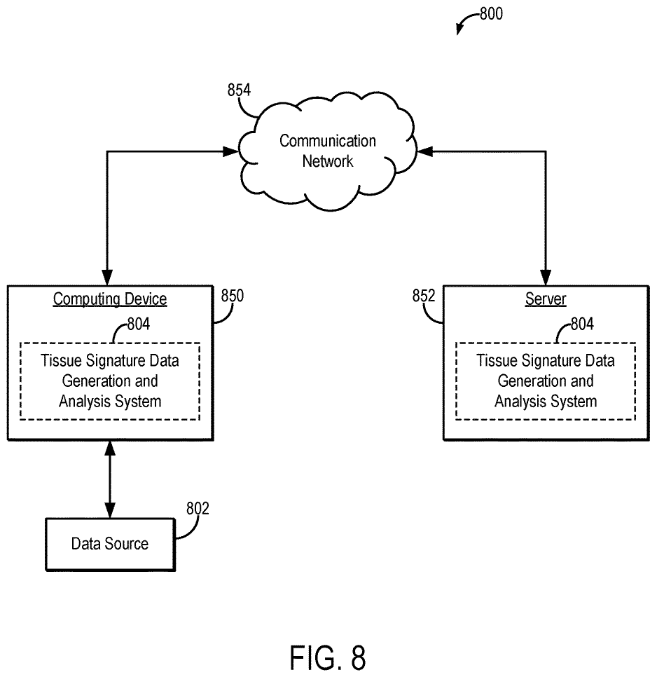

FIG. 8 shows an example of a system 800 for assessing tissue damage (e.g., tissue damage extent and distribution) in accordance with some embodiments described in the present disclosure. As shown in FIG. 8, a computing device 850 can receive one or more types of data (e.g., medical image data, tissue signature data) from data source 802. In some embodiments, computing device 850 can execute at least a portion of a tissue signature data generation and analysis system 804 to generate and analyze tissue signature data from medical image data received from the data source 802.

Additionally or alternatively, in some embodiments, the computing device 850 can communicate information about data received from the data source 802 to a server 852 over a communication network 854, which can execute at least a portion of the tissue signature data generation and analysis system 804. In such embodiments, the server 852 can return information to the computing device 850 (and/or any other suitable computing device) indicative of an output of the tissue signature data generation and analysis system 804.

In some embodiments, computing device 850 and/or server 852 can be any suitable computing device or combination of devices, such as a desktop computer, a laptop computer, a smartphone, a tablet computer, a wearable computer, a server computer, a virtual machine being executed by a physical computing device, and so on. The computing device 850 and/or server 852 can also reconstruct images from the data.

In some embodiments, data source 802 can be any suitable source of data (e.g., measurement data, images reconstructed from measurement data, processed image data), such as a medical imaging system, another computing device (e.g., a server storing measurement data, images reconstructed from measurement data, processed image data), and so on. In some embodiments, data source 802 can be local to computing device 850. For example, data source 802 can be incorporated with computing device 850 (e.g., computing device 850 can be configured as part of a device for measuring, recording, estimating, acquiring, or otherwise collecting or storing data). As another example, data source 802 can be connected to computing device 850 by a cable, a direct wireless link, and so on. Additionally or alternatively, in some embodiments, data source 802 can be located locally and/or remotely from computing device 850, and can communicate data to computing device 850 (and/or server 852) via a communication network (e.g., communication network 854).

In some embodiments, communication network 854 can be any suitable communication network or combination of communication networks. For example, communication network 854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 854 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, and so on.

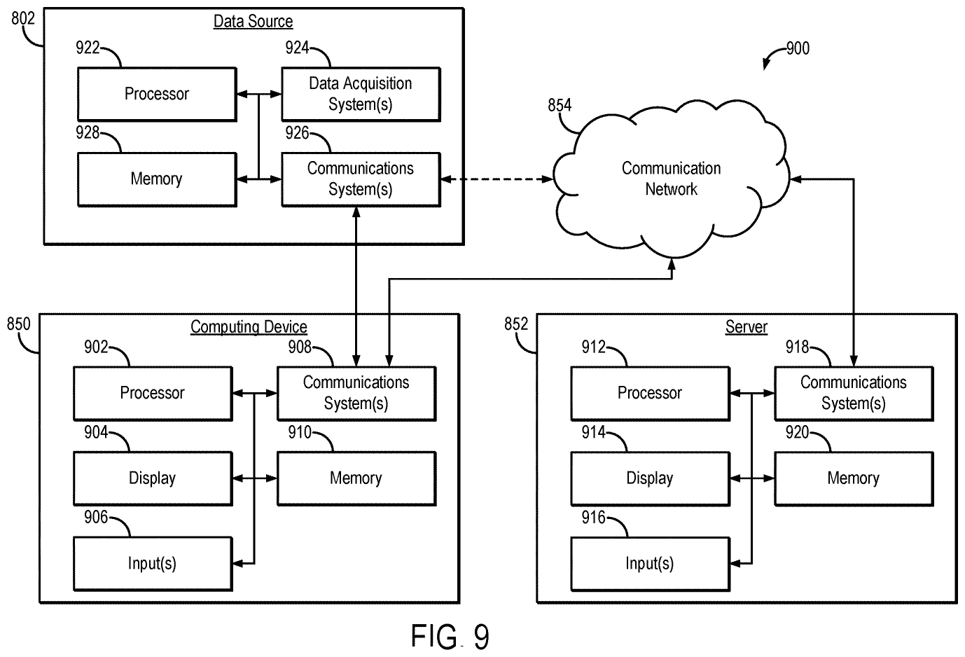

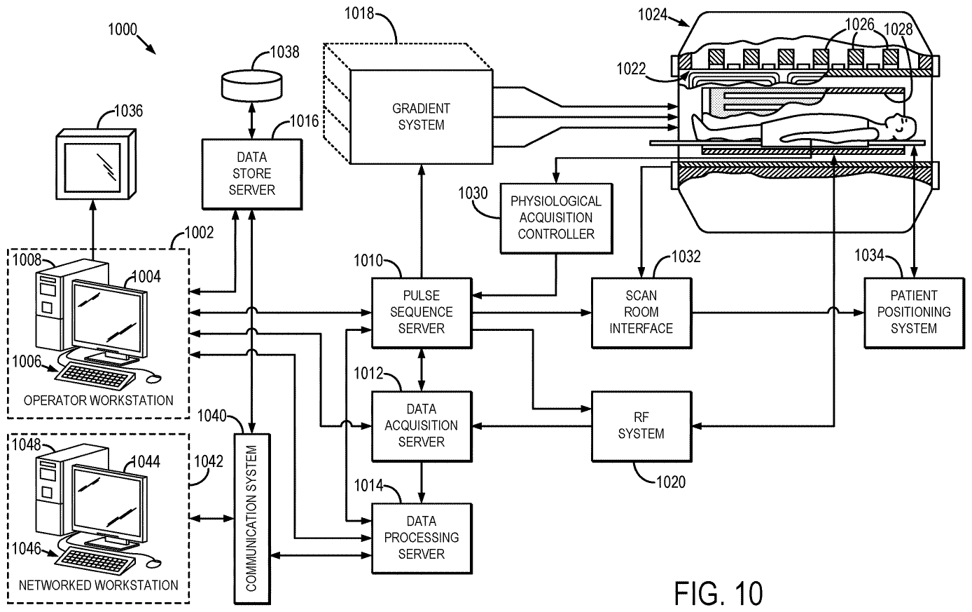

Referring now to FIG. 9, an example of hardware 900 that can be used to implement data source 802, computing device 850, and server 852 in accordance with some embodiments of the systems and methods described in the present disclosure is shown.

As shown in FIG. 9, in some embodiments, computing device 850 can include a processor 902, a display 904, one or more inputs 906, one or more communication systems 908, and/or memory 910. In some embodiments, processor 902 can be any suitable hardware processor or combination of processors, such as a central processing unit (CPU), a graphics processing unit (GPU), and so on. In some embodiments, display 904 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 906 can include any suitable input devices and/or sensors that can be used to receive user input, such as a keyboard, a mouse, a touchscreen, a microphone, and so on.

In some embodiments, communications systems 908 can include any suitable hardware, firmware, and/or software for communicating information over communication network 854 and/or any other suitable communication networks. For example, communications systems 908 can include one or more transceivers, one or more communication chips and/or chip sets, and so on. In a more particular example, communications systems 908 can include hardware, firmware, and/or software that can be used to establish a Wi-Fi connection, a Bluetooth connection, a cellular connection, an Ethernet connection, and so on.

In some embodiments, memory 910 can include any suitable storage device or devices that can be used to store instructions, values, data, or the like, that can be used, for example, by processor 902 to present content using display 904, to communicate with server 852 via communications system(s) 908, and so on. Memory 910 can include any suitable volatile memory, non-volatile memory, storage, or any suitable combination thereof. For example, memory 910 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 910 can have encoded thereon, or otherwise stored therein, a computer program for controlling operation of computing device 850. In such embodiments, processor 902 can execute at least a portion of the computer program to present content (e.g., images, user interfaces, graphics, tables), receive content from server 852, transmit information to server 852, and so on. For example, the processor 902 and the memory 910 can be configured to perform the methods described herein (e.g., the method of FIG. 1, the method of FIG. 2, the method of FIG. 3).

In some embodiments, server 852 can include a processor 912, a display 914, one or more inputs 916, one or more communications systems 918, and/or memory 920. In some embodiments, processor 912 can be any suitable hardware processor or combination of processors, such as a CPU, a GPU, and so on. In some embodiments, display 914 can include any suitable display devices, such as an LCD screen, LED display, OLED display, electrophoretic display, a computer monitor, a touchscreen, a television, and so on. In some embodiments, inputs 916 can include any suitable input devices and/or sensors that can be used to receive user input, such as a keyboard, a mouse, a touchscreen, a microphone, and so on.

In some embodiments, communications systems 918 can include any suitable hardware, firmware, and/or software for communicating information over communication network 854 and/or any other suitable communication networks. For example, communications systems 918 can include one or more transceivers, one or more communication chips and/or chip sets, and so on. In a more particular example, communications systems 918 can include hardware, firmware, and/or software that can be used to establish a Wi-Fi connection, a Bluetooth connection, a cellular connection, an Ethernet connection, and so on.

In some embodiments, memory 920 can include any suitable storage device or devices that can be used to store instructions, values, data, or the like, that can be used, for example, by processor 912 to present content using display 914, to communicate with one or more computing devices 850, and so on. Memory 920 can include any suitable volatile memory, non-volatile memory, storage, or any suitable combination thereof. For example, memory 920 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 920 can have encoded thereon a server program for controlling operation of server 852. In such embodiments, processor 912 can execute at least a portion of the server program to transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone), and so on.

In some embodiments, the server 852 is configured to perform the methods described in the present disclosure. For example, the processor 912 and memory 920 can be configured to perform the methods described herein (e.g., the method of FIG. 1, the method of FIG. 2, the method of FIG. 3).

In some embodiments, data source 802 can include a processor 922, one or more data acquisition systems 924, one or more communications systems 926, and/or memory 928. In some embodiments, processor 922 can be any suitable hardware processor or combination of processors, such as a CPU, a GPU, and so on. In some embodiments, the one or more data acquisition systems 924 are generally configured to acquire data, images, or both, and can include a medical imaging system. Additionally or alternatively, in some embodiments, the one or more data acquisition systems 924 can include any suitable hardware, firmware, and/or software for coupling to and/or controlling operations of a medical imaging system. In some embodiments, one or more portions of the data acquisition system(s) 924 can be removable and/or replaceable.

Note that, although not shown, data source 802 can include any suitable inputs and/or outputs. For example, data source 802 can include input devices and/or sensors that can be used to receive user input, such as a keyboard, a mouse, a touchscreen, a microphone, a trackpad, a trackball, and so on. As another example, data source 802 can include any suitable display devices, such as an LCD screen, an LED display, an OLED display, an electrophoretic display, a computer monitor, a touchscreen, a television, etc., one or more speakers, and so on.

In some embodiments, communications systems 926 can include any suitable hardware, firmware, and/or software for communicating information to computing device 850 (and, in some embodiments, over communication network 854 and/or any other suitable communication networks). For example, communications systems 926 can include one or more transceivers, one or more communication chips and/or chip sets, and so on. In a more particular example, communications systems 926 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 928 can include any suitable storage device or devices that can be used to store instructions, values, data, or the like, that can be used, for example, by processor 922 to control the one or more data acquisition systems 924, and/or receive data from the one or more data acquisition systems 924; to generate images from data; present content (e.g., data, images, a user interface) using a display; communicate with one or more computing devices 850; and so on. Memory 928 can include any suitable volatile memory, non-volatile memory, storage, or any suitable combination thereof. For example, memory 928 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 928 can have encoded thereon, or otherwise stored therein, a program for controlling operation of data source 802. In such embodiments, processor 922 can execute at least a portion of the program to generate images, transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

Referring particularly now to FIG. 10, an example of an MRI system 1000 that can implement the methods described here is illustrated. The MRI system 1000 includes an operator workstation 1002 that may include a display 1004, one or more input devices 1006 (e.g., a keyboard, a mouse), and a processor 1008. The processor 1008 may include a commercially available programmable machine running a commercially available operating system. The operator workstation 1002 provides an operator interface that facilitates entering scan parameters into the MRI system 1000. The operator workstation 1002 may be coupled to different servers, including, for example, a pulse sequence server 1010, a data acquisition server 1012, a data processing server 1014, and a data store server 1016. The operator workstation 1002 and the servers 1010, 1012, 1014, and 1016 may be connected via a communication system 1040, which may include wired or wireless network connections.

The pulse sequence server 1010 functions in response to instructions provided by the operator workstation 1002 to operate a gradient system 1018 and a radiofrequency (“RF”) system 1020. Gradient waveforms for performing a prescribed scan are produced and applied to the gradient system 1018, which then excites gradient coils in an assembly 1022 to produce the magnetic field gradients Gₓ, G_{y}, and G_{z} that are used for spatially encoding magnetic resonance signals. The gradient coil assembly 1022 forms part of a magnet assembly 1024 that includes a polarizing magnet 1026 and a whole-body RF coil 1028.

RF waveforms are applied by the RF system 1020 to the RF coil 1028, or a separate local coil to perform the prescribed magnetic resonance pulse sequence. Responsive magnetic resonance signals detected by the RF coil 1028, or a separate local coil, are received by the RF system 1020. The responsive magnetic resonance signals may be amplified, demodulated, filtered, and digitized under direction of commands produced by the pulse sequence server 1010. The RF system 1020 includes an RF transmitter for producing a wide variety of RF pulses used in MRI pulse sequences. The RF transmitter is responsive to the prescribed scan and direction from the pulse sequence server 1010 to produce RF pulses of the desired frequency, phase, and pulse amplitude waveform. The generated RF pulses may be applied to the whole-body RF coil 1028 or to one or more local coils or coil arrays.

The RF system 1020 also includes one or more RF receiver channels. An RF receiver channel includes an RF preamplifier that amplifies the magnetic resonance signal received by the coil 1028 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 1010 may receive patient data from a physiological acquisition controller 1030. By way of example, the physiological acquisition controller 1030 may receive signals from a number of different sensors connected to the patient, including electrocardiograph (ECG) signals from electrodes, or respiratory signals from a respiratory bellows or other respiratory monitoring devices. These signals may be used by the pulse sequence server 1010 to synchronize, or “gate,” the performance of the scan with the subject's heart beat or respiration.

The pulse sequence server 1010 may also connect to a scan room interface circuit 1032 that receives signals from various sensors associated with the condition of the patient and the magnet system. Through the scan room interface circuit 1032, a patient positioning system 1034 can receive commands to move the patient to desired positions during the scan.

The digitized magnetic resonance signal samples produced by the RF system 1020 are received by the data acquisition server 1012. The data acquisition server 1012 operates in response to instructions downloaded from the operator workstation 1002 to receive the real-time magnetic resonance data and provide buffer storage, so that data is not lost by data overrun. In some scans, the data acquisition server 1012 passes the acquired magnetic resonance data to the data processor server 1014. In scans that require information derived from acquired magnetic resonance data to control the further performance of the scan, the data acquisition server 1012 may be programmed to produce such information and convey it to the pulse sequence server 1010. For example, during pre-scans, magnetic resonance data may be acquired and used to calibrate the pulse sequence performed by the pulse sequence server 1010. As another example, navigator signals may be acquired and used to adjust the operating parameters of the RF system 1020 or the gradient system 1018, or to control the view order in which k-space is sampled. In still another example, the data acquisition server 1012 may also process magnetic resonance signals used to detect the arrival of a contrast agent in a magnetic resonance angiography (MRA) scan. For example, the data acquisition server 1012 may acquire magnetic resonance data and processes it in real-time to produce information that is used to control the scan.

The data processing server 1014 receives magnetic resonance data from the data acquisition server 1012 and processes the magnetic resonance data in accordance with instructions provided by the operator workstation 1002. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 1014 are conveyed back to the operator workstation 1002 for storage. Real-time images may be stored in a data base memory cache, from which they may be output to operator display 1002 or a display 1036. Batch mode images or selected real time images may be stored in a host database on disc storage 1038. When such images have been reconstructed and transferred to storage, the data processing server 1014 may notify the data store server 1016 on the operator workstation 1002. The operator workstation 1002 may be used by an operator to archive the images, produce films, or send the images via a network to other facilities.

The MRI system 1000 may also include one or more networked workstations 1042. For example, a networked workstation 1042 may include a display 1044, one or more input devices 1046 (e.g., a keyboard, a mouse), and a processor 1048. The networked workstation 1042 may be located within the same facility as the operator workstation 1002, or in a different facility, such as a different healthcare institution or clinic.

The networked workstation 1042 may gain remote access to the data processing server 1014 or data store server 1016 via the communication system 1040. Accordingly, multiple networked workstations 1042 may have access to the data processing server 1014 and the data store server 1016. In this manner, magnetic resonance data, reconstructed images, or other data may be exchanged between the data processing server 1014 or the data store server 1016 and the networked workstations 1042, such that the data or images may be remotely processed by a networked workstation 1042.

The present disclosure has described one or more preferred embodiments, and it should be appreciated that many equivalents, alternatives, variations, and modifications, aside from those expressly stated, are possible and within the scope of the disclosure.

1. A method for assessing tissue damage in a subject using medical imaging, the method comprising:
(a) accessing medical image data with a computer system, wherein the medical image data comprise medical images acquired from a subject, wherein the medical images include a calibration region and a target region;
(b) processing the medical image data with the computer system to generate tissue signature data comprising one or more probabilistic signature profiles that indicate at least one of an extent of tissue damage or distribution patterns associated with the tissue damage; and
(c) generating, with the computer system, a report that indicates an assessment of tissue damage based on the tissue signature data.

2. The method of claim 1, wherein the tissue signature data comprise a cross-disparity signature profile between the calibration region and the target region.

3. The method of claim 2, wherein the cross-disparity signature profile is generated by:
computing cross-disparity values between voxels in the calibration region and voxels in the target region; and
generating the cross-disparity signature profile by computing a probability density function of the cross-disparity values.

4. The method of claim 3, wherein the probability density function is a normalized histogram.

5. The method of claim 3, wherein the cross-disparity values are normalized to a unified range before generating the cross-disparity signature profile.

6. The method of any one of claim 1 or 2, wherein the tissue signature data comprise a co-disparity signature profile between the target region and itself.

7. The method of claim 6, wherein the co-disparity signature profile is generated by:
computing co-disparity values between voxels in the target region with other voxels in the target region; and
generating the co-disparity signature profile by computing a probability density function of the co-disparity values.

8. The method of claim 7, wherein the probability density function is a normalized histogram.

9. The method of claim 7, wherein the co-disparity values are normalized to a unified range before generating the co-disparity signature profile.

10. The method of claim 6, wherein the tissue signature data comprise a tissue damage signature index that quantitatively encodes the tissue damage extent and the distribution pattern associated with the tissue damage.

11. The method of claim 10, wherein the tissue signature data include both the cross-disparity profile and the co-disparity profile and the tissue damage signature index is computed using both the cross-disparity signature profile and the co-disparity signature profile.

12. The method of claim 11, wherein the tissue damage signature index is computed as a ratio between a first distance metric calculated using the cross-disparity signature profile and a second distance metric calculated using the cross-disparity signature profile and the co-disparity signature profile.

13. The method of claim 12, wherein the first distance metric is calculated by splitting the cross-disparity signature profile into a first cross-disparity signature profile and a second cross-disparity signature profile and computing a distance between the first cross-disparity signature profile and the second cross-disparity signature profile.

14. The method of claim 13, wherein the splitting the cross-disparity signature profile into the first cross-disparity signature profile and second cross-disparity signature profile comprises identifying a peak in the cross-disparity signature profile and splitting the cross-disparity signature profile into the first cross-disparity signature profile and the second cross-disparity signature profile relative to the identified peak.

15. The method of claim 14, wherein the peak comprises a maximum point in the cross-disparity signature profile.

16. The method of claim 12, wherein the second distance metric comprises computing a distance between the cross-disparity signature profile and the co-disparity signature profile.

17. The method of claim 6, wherein the tissue signature data comprise a distance computed between the cross-disparity signature profile and the co-disparity signature profile.

18. The method of claim 6, wherein the cross-disparity signature profile comprises a first split-peak cross-disparity signature profile and a second split-peak cross-disparity signature profile and the tissue signature data comprise a distance computed between the first split-peak cross-disparity signature profile and the second split-peak cross-disparity signature profile.

19. The method of claim 18, wherein the cross-disparity signature profile is split into the first split-peak cross-disparity signature profile and second split-peak cross-disparity signature profile by identifying a peak in the cross-disparity signature profile and splitting the cross-disparity signature profile into the first split-peak cross-disparity signature profile and the second split-peak cross-disparity signature profile relative to the identified peak.

20. The method of any one of claim 13 or 16-19, wherein the distance is a chi square distribution distance.

21. The method of claim 1, wherein the calibration region comprises a blood pool and the target region comprises myocardial wall tissue.

22. The method of claim 21, wherein the tissue signature data comprise a cross-disparity signature profile between myocardial wall tissue and blood pool in the medical image data; a co-disparity signature profile of the myocardial wall tissue in the medical image data, wherein the co-disparity signature profile encodes a composition of image intensity distribution patterns in the entire myocardial wall; and a scar burden signature index that encodes both scar extent and distribution patterns simultaneously based on probability density distribution differences of the cross-disparity signature profile and the co-disparity signature profile.

23. The method of claim 21, wherein the medical image data comprise late gadolinium enhancement (LGE) magnetic resonance images acquired using a magnetic resonance imaging system.

24. The method of claim 1, wherein the medical image data comprise magnetic resonance images acquired with a magnetic resonance imaging (MRI) system.

25. The method of claim 1, wherein the medical image data comprise computed tomography (CT) images acquired with a CT system.

26. The method of claim 1, wherein the medical image data comprise positron emission tomography (PET) images acquired with a PET system.

27. The method of claim 26, wherein the target region comprises a region of radiotracer uptake and the calibration region comprises a region with less radiotracer uptake than the target region.

28. The method of claim 1, wherein the report indicates the extent of tissue damage and distribution patterns associated with the tissue damage.

29. The method of claim 1, wherein the report indicates a severity of a disease in the subject.

30. The method of claim 29, wherein the disease comprises a heart disease.

31. The method of claim 30, wherein the heart disease comprises a least one of myocardial infarction, myocarditis, arrhythmia, ventricular tachycardia, atrial fibrillation, cardiac amyloidosis, or cardiomyopathy.

32. The method of claim 1, wherein the report indicates a treatment for the tissue damage in the subject.

33. The method of claim 1, wherein the report indicates whether the subject is at risk for an adverse health event based on the tissue signature data.

34. The method of claim 1, wherein the report indicates a disease progression in the subject.

35. The method of claim 1, wherein generating the report comprises:
accessing a machine learning model with the computer system, wherein the machine learning model has been trained on training data to assess a disease condition based on tissue signature data;
inputting the tissue signature data to the machine learning model, generating classified feature data as an output; and
outputting the classified feature data as part of the report.

36. The method of claim 35, wherein the classified feature data comprise a risk score indicating a likelihood of the subject suffering from a particular disease condition.

37. The method of claim 35, wherein the classified feature data comprise a probability for a particular classification of a disease condition.

38. The method of claim 35, wherein the classified feature data indicate a differentiation between different disease conditions in the subject.

39. The method of claim 35, wherein the classified feature data indicate a severity of a disease condition in the subject.
